(19) United States
(12) Patent Application Publication
(10) Pub. No.: US 2010/0139655 A1
(43) Pub. Date: Jun. 10, 2010

(54) DRY-POWDER INHALER

(75) Inventors: Amir Genosar, Boulder, CO (US); Amnon Kritzman, Karkur (IL); Dan Adler, Karkur (IL)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Aespironics Ltd, Omer (IL)

(21) Appl. No.: 12/515,108

(22) PCT Filed: Nov. 12, 2007
(86) PCT No.: PCT/IL07/01386

§ 371 Date: Jan. 8, 2010

(30) Foreign Application Priority Data
Nov. 19, 2006 (IL) 179,387
Nov. 6, 2007 (IL) 187,201

(51) Int. Cl. A61M 15/00

(52) U.S. Cl. 128/203.15

(57) ABSTRACT

A dry-powder, breath-powered inhaler device comprising at least one air inlet, a flow chamber and an air outlet leading to a mouthpiece, the flow chamber further comprising at least one drug-containing volume and at least one scraping means and a rotor assembly comprising an axis and at least one blade; wherein the inhalation action of the patient applied at the air outlet causes air to enter the at least one air inlet such that the air interacts with the at least one rotor blade causing the rotor to rotate and thus generate breath-driven, relative motion between the at least one drug-containing volume and the at least one scraping means, such that fine particles of powder are released from the drug-containing volume; the arrangement being such that the air exits the flow chamber via the periphery of the flow chamber, carrying out the fine particles to the outlet to be inhaled by the patient.

### FIELD OF THE INVENTION

This invention relates to a medical device for dry-powder drug inhalation. Specifically, the present invention is a single-step inhaler where the act of inhalation scrapes powder from a drug-containing volume so that this powder is inhaled into the lungs.

### BACKGROUND OF THE INVENTION

Numerous drugs, medications and other substances are inhaled into the lungs for rapid absorption in the blood stream. Inhaled drugs fall into two main categories: (1) liquids, including suspensions; and (2) powders. The present invention relates to the latter category.

Dry-powder inhalers need to deliver a particle size that is predominantly below 5 microns for maximum effectiveness. Such small particles are, however, thermodynamically unstable due to their high surface area to volume ratio, which provides significant excess surface free energy and encourages particles to agglomerate. In the inhaler, agglomeration of small particles and adherence of particles to the walls of the inhaler are problems that result in the active particles leaving the inhaler as large agglomerates or being unable to leave the inhaler and remaining adhered to the interior of the inhaler.

The following prior art approaches to this problem describe the production of the requisite powder particle size by means of scraping from a solid drug matrix or compressed powder, where a separate unit is powered in order to accomplish this scraping. U.S. Pat. No. 5,347,999 describes an inhaler device in which the medicinal substance is stored in a solid form and the required dose is scraped into a powder form immediately before the inhalation process by a mechanical operation such as abrasion by means of a brush. U.S. Pat. No. 5,617,845 describes an inhaler device in which metering of the dose to be delivered is carried out by means of a specially shaped metering notch which is rotated past a slightly compressed-powder charge, and in which a trigger-operated pump is primed manually before the inhalation process by means of a button. U.S. Pat. No. 5,887,586 describes a dry-powder aerosol generator, which is connected to a removable nose mask via a conduit system, where the aerosol generator comprises a scraping mechanism, by means of which powder can be scraped off a tablet of compressed-powder, together with a means for aerosolizing the scraped-off powder in an air flow. U.S. Pat. No. 6,012,454 describes an inhaler which detects the patient's inspiration and triggers a deagglomeration/aerosolization means which operates to ensure efficient aerosolization of the medicament in the air stream. WO 03/092774 describes an inhaler which includes a unit for producing powder from a solid monotropic material using an abrasive element or scraper.

However, in none of these prior art devices is the scraping actually powered by, and therefore optimally synchronized with, the inhalation action of the patient. On the contrary, said devices generally release the scraped powder either at one point during the inhalation process (such as at its start) or all the time with no relationship to the actual air intake by the patient. This results in a complex mechanism which, being poorly synchronized, presents additional opportunities for the powder particles to aggregate.

U.S. Pat. No. 6,871,647 describes an inhaler in which a mesh is incorporated into the drug powder compartments and where the drug powder is entrained purely by the air flow through said mesh. Similarly, U.S. Pat. No. 5,388,572 describes a mesh disc impregnated with drug powder doses, but where the air flow is produced by a piston which produces an air pressure blast. However, although both of these approaches incorporate a mesh in the encapsulation of the powder dose, in both cases the entraining mechanism is purely the air flow passing through the mesh, and no rotor is employed to rattle, beat, vibrate or scrape the mesh.

In view of these drawbacks and limitations of the prior art, what is needed is a simple and inexpensive inhaler without complex mechanical or electronic powder generators, capable of consistently delivering predominantly sub 5 micron particle sizes.

Therefore, it is an object of the invention to provide a simple, breath-powered inhaler where the act of inhalation itself drives the process causing the dry-powder to be released from a drug-containing volume.

It is a still further object of the invention to provide a dry-powder inhaler which synchronizes the drug release with the inhalation action of the patient, by spreading the delivery over a defined duration of the breath.

It is a further object of the invention to provide a convenient and portable housing for said inhaler.

It is a still further object of the invention to provide said specially designed device in a credit-card format.

It is a still further object of the invention to provide an ergonomic mouthpiece for miniature device, where said mouthpiece can be stored within a credit-card format device.

It is further the object of the invention to provide a device that enables the transporting of the drug separate from the device such that the patient can load said drug into the device.

It is further the object of the invention to provide a device that is indifferent to accidental air-blow into the device.

These and other objects of the present invention are achieved in the preferred embodiments disclosed below by providing a breath-powered dry-powder inhaler.

### SUMMARY OF THE INVENTION

The inhaler device of the present invention provides an improved and simplified mechanism for dry-powder drug inhalation, which ensures the synchronization of fine-particle release during inhalation. The operating principle of said device is that the act of inhalation itself causes fine powder to be scratched, scraped, rubbed, brushed away or otherwise removed from a drug-containing volume due to the impact of a rotor, where the thus released powder is inhaled directly. Advantageously, such an approach is inherently free of the problems of prior art devices where a powder dose can be spilled or where exhaling into the device can disturb the powder. As the powder for inhalation is only produced during the inhalation, the synchronization of the powder inhalation with the breath is achieved inherently in this design. Depending on the drug type, said synchronization with the inhalation curve is extremely important in order to ensure that the drug is delivered to the required areas of the lungs. Thus, for several drugs, a too-early or too-late delivery results in extremely low efficiency of the administration, which in turn can affect the results of the treatment and even limit the use of certain devices from critical drugs. Additionally, in many cases, a pre-determined delay of the drug discharge to a certain point in the inhalation curve and the release of the drug over a defined period of that inhalation curve (rather than in a bolus) provides optimal results. By having the drug in a solid form in the device, the management of the drug in the device becomes simpler and thus enables a simpler and more compact mechanism.

The drug-containing volume of the present invention shall refer to any form of drug, vaccine or other therapeutic agent in which a powder, micronized powder or microspheres is (a) formed into a solid matrix; (b) impregnated onto a surface such as a plastic; (c) embedded in a carrier like a textured material such as a fabric or other fibrous material, (d) held behind a mesh, or (e) deposited on a surface. Said forming may be by means of compression, compression with an excipient, electro-static deposition, evaporation, drying or any other means of forming a solid matrix known in the art. Similarly, said impregnation or embedding of powders or microspheres may be by any means known in the art.

Likewise, the free-powder releasing entailed in the present invention shall refer to any form of scratching, scraping, rubbing, rattling, or brushing of the powder from said drug-containing volume caused by the repeated impaction between the drug-containing volume and the scraping means.

The inhaler device of the present invention is a breath-powered inhaler device comprising at least one air inlet, a flow chamber and an air outlet leading to a mouthpiece, said flow chamber further comprising at least one drug-containing volume and at least one free-powder releasing means for entraining powder into the airflow through said device and a rotor assembly comprising an axis and at least one rotor blade; such as a turbine blade, wherein the inhalation action of the patient applied at said air outlet causes air to enter said at least one air inlet such that said air interacts with said at least one rotor blade causing said rotor to rotate and thus generate breath-driven, relative motion between said at least one drug-containing volume and said at least one free-powder releasing means, such that fine free particles of powder are released from said drug-containing volume; the arrangement being such that the air exits said flow chamber via the periphery of said flow chamber, carrying out said fine particles to said outlet to be inhaled by the patient.

In preferred embodiments of the present invention said free-powder releasing means can be in the form of a scraper, a brush, a beater or a similar means for releasing free, fine powder from said drug-containing volume.

In preferred embodiments of the present invention said free-powder releasing means is selected from the group consisting of a scraping means, a brush and a beater means.

In a preferred embodiment said scraping means is located either at the distal end of said rotor blade where said blade gradually extends outwards as said rotor rotates, or at the distal end of an arm attached to said rotor blade where said arm gradually extending outwards as said rotor rotates. In a further embodiment, said scraping means is fixed and said drug-containing volume is attached to the rotor assembly. Said drug-containing volume comprises a medicinal agent belonging to the group including drugs, vaccines and other therapeutic agents. Said drug-containing volume comprises fine powder, micronized powder and/or microspheres; and is formed by compression into a solid matrix, impregnation onto a surface, embedding in a carrier, holding behind a mesh or deposition on to a surface.

In preferred embodiments of the present invention, said dry-powder inhaler device comprises a multiplicity of air inlets.

Preferably the scraping means is the tip of a rotor arm or the tip of an arm attached to a rotor, said rotor being a turbine powered by the inhalation flow. As said rotor rotates, at least part of said arm gradually extends outwards such that its tip serves to scrape a drug-containing volume. This arrangement ensures a time lag between the start of said inhalation action and the first release of said fine particles.

Said device preferably further comprises (a) a particle filter located between said flow chamber and said outlet to ensure that large particles are not inhaled and (b) a mouthpiece. Said mouthpiece may either be an integral part of said inhaler device or may be attached by the patient to said outlet. In the latter case, the inhaler device may further comprise a storage compartment for said mouthpiece.

The inhaler device is preferably shaped like a credit-card or like a conventional hand-held inhaler. Alternatively it may have any other ergonomically suitable shape, including that of a cylinder, a prism, a disk, and an oval.

The invention will now be described in connection with certain preferred embodiments with reference to the following illustrative figures so that it may be more fully understood.

With specific reference now to the figures in detail, it is stressed that the particulars shown are by way of example and for purposes of illustrative discussion of the preferred embodiments of the present invention only and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

### BRIEF DESCRIPTION OF THE DRAWINGS

FIG. 1 presents isometric and planar views of a single-use disposable credit-card shape embodiment of the present invention;

FIG. 2 shows planar views showing the scraping action performed by the rotor of the above embodiment;

FIG. 3 presents planar views of a further credit-card shaped embodiment of the invention where an alternative turbine design is employed; and

FIG. 4 shows isometric and planar views of a hand-held embodiment of the present invention showing the impingement of the airflow and the scraping of the drug by the blade tips in this configuration. The planar view has a cut-away section so as to expose the internal parts of the embodiment.

FIG. 5 provides isometric exploded, isometric and planar views of a preferred embodiment of the drug-containing volume of the present invention where said volume is fabricated in sandwich form using two meshes connected within a semi-rigid framework;

FIG. 6 provides planar views of the inhalation device of the present invention showing a preferred embodiment of the placement of said sandwich form of said drug-containing volume, so as to illustrate the beating action of a rotor blade against such volume;

FIG. 7 provides a similar planar view of an embodiment of the inhalation device, where said drug-containing volume is placed in a further preferred orientation; and

FIG. 8 provides an isometric view of the drug-containing volume, fabricated as a mesh cylinder.

### DETAILED DESCRIPTION OF THE DRAWINGS

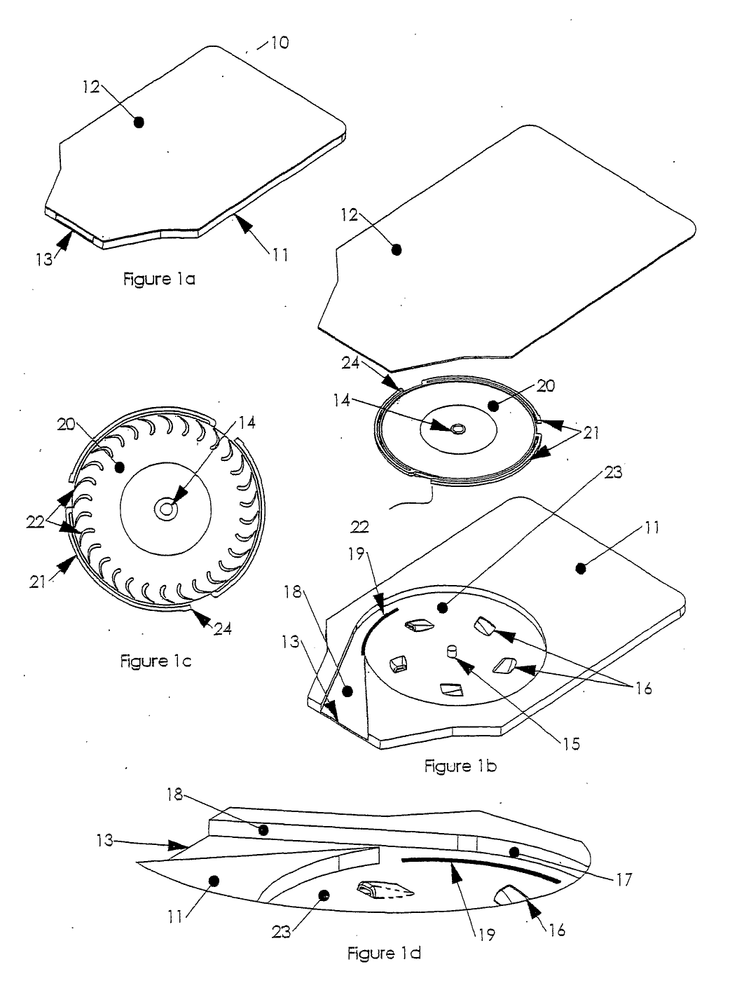

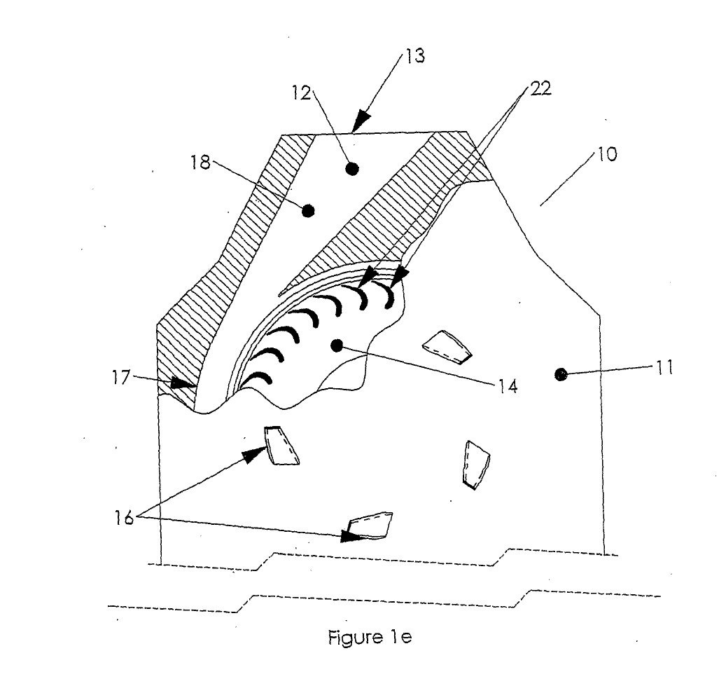

FIG. 1 shows a preferred embodiment of the device of the present invention in which a credit-card style design is employed. FIG. 1a provides an overall isometric view of this preferred embodiment. In this embodiment, the device 10 has the form-factor of a credit-card which, advantageously, is simple and convenient to store in a wallet or pocket. The outer envelope or housing of the device is formed by a base part 11 (herein “spine”), and a thin film wall 12. Said film wall 12 may comprise a multi-layer plastic film and/or a metalized plastic film, and/or a metal foil. Advantageously, this construction enables the device 10 to benefit from the strength and excellent barrier properties that such film walls possess. The spine 11 incorporates the flow path features (as described further below) and is preferably an injection-molded part. The thin film wall 12 seals onto the spine 11 such that it completes the internal flow chamber and the flow path, leaving an outlet port 13 open in the mouthpiece section of the device 10. Said outlet port 13 further comprises a particulate filter (not shown) as described in co-pending application PCT/IL2006/000647, hereby incorporated by reference. Said particulate filter prevents large size powder particles from reaching the patient.

Referring now to FIG. 1b, a perspective exploded view of said preferred embodiment is provided, showing the air inlets 16, the air outlet channel 18 leading to the outlet port 13, the drug-containing volume 19 and the axis 15 for mounting the rotor 20. The rotor assembly 20 is mounted into the flow chamber 23, such that air jets will enter this flow chamber 23 via the air inlets 16 in a direction tangential to the axis of rotation. In this preferred embodiment, the rotor 20 comprises a relatively flat disk with a central hole 14 (for engaging with the axis 15), flexible arms 21 and rotor blades 22. Note that the blades 22 of the rotor 20 are not visible in FIG. 1b as they are located on the other side of the rotor 20, facing the air inlets 16. The flexible arms 21 have a proximal end attached to the periphery of the rotor 15, while the distal end 24, which constitutes the scraping means, is free to extend away from the center of the rotor assembly 20. The rotation of the rotor 20 causes centrifugal displacement of said flexible arms 21, such that their distal ends 24 extend out and scrape the drug-containing volume 19. FIG. 1c shows the underside of the rotor 20, illustrating the location of the blades 22.

Referring now to FIG. 1d, a close-up of the flow chamber 23 shows the orientation of the air inlets 16 into this chamber, showing more clearly how they impart rotational force to the rotor 20. Also shown more clearly in this figure is the low-profile nature of drug-containing volume 19 mounted or otherwise attached to the spine 13. This low-profile nature is what enables the scraping means to pass over this volume repeatedly. As it does so, said scraping means scrapes, rattles, rubs, brushes or otherwise removes some of the fine powder from it each time. The raffling of the drug-containing volume 19 may be achieved by part of the rotor assembly 20 interacting with a non-flat part of the flow chamber 23. In this preferred embodiment, the drug-containing means 19 is located close to the air outlet channel 18 so as to minimize the time and the path between the scraping of the particles and their inhalation by the patient. Advantageously this minimizes the chance for such particles to aggregate, while at the same time minimizing the proportion of the dose that can become trapped inside the device 10.

Referring now to FIG. 1e, a cut-away planar view of the underside of the device 10 is shown, showing the inlet nozzles 16 and the blades 22 of the rotor 20. The inlet nozzles 16 are clearly seen to be positioned on an imaginary circle having a radius smaller than the radius of the circle of the blade's leading edge. The outlet volute 17 (i.e. a curved funnel which gradually increases its cross-sectional area as it approaches its outlet) leads the air to an outlet channel 18. This construction is irregular for most power generation turbo machines, where regularly the air flow is from the periphery of the rotor toward its center, but is required in this embodiment in order to facilitate the air flow to pass from the rotor housing to the mouthpiece in a low-profile device such as the credit-card-shape device 10 of this preferred embodiment. The air outlet channel 18 can possibly be designed as a diffuser to slow the flow velocity without causing major turbulence and pressure loss. Note that the blades 22 have a shape where the leading edge is facing the direction of rotation and the trailing edge is pulled strongly backward, in order to cause a significant reduction of the absolute velocity tangential component, such that at the rotor exit it vanishes completely (i.e., is equal to zero). Thus the exit vortex is eliminated, which reduces flow losses and improves the exit flow field.

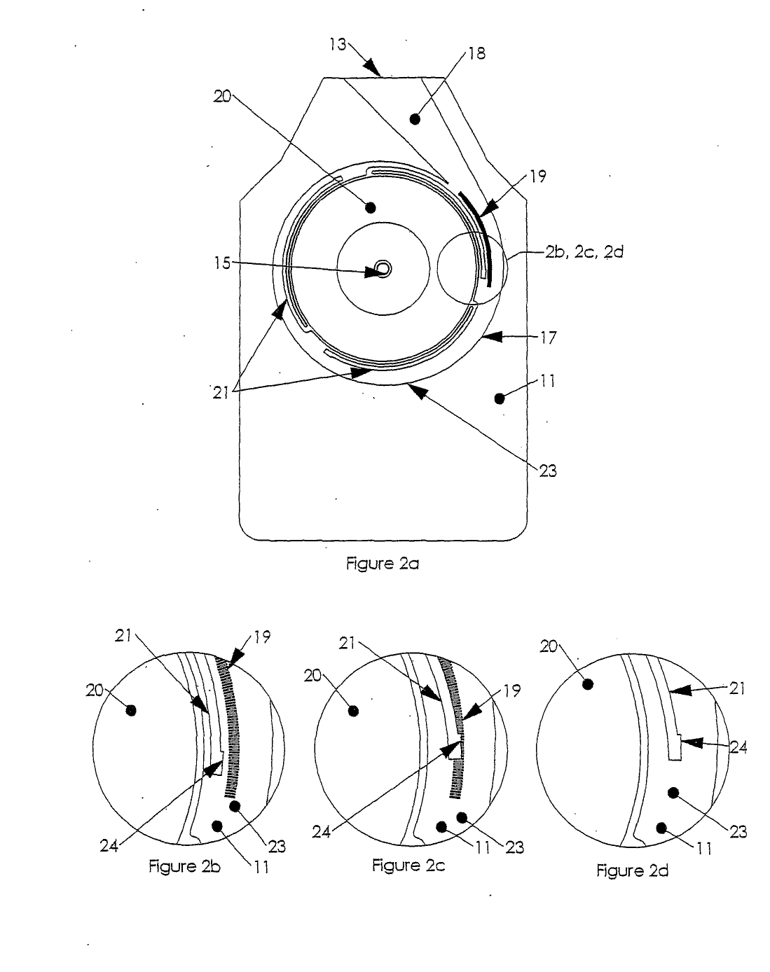

Referring now to FIG. 2, the operation of the rotor 20 and its interaction with the drug-containing volume 19 during inhalation is shown. In these figures, a series of enlarged planar views of the flexible arms 21 of the rotor 20 are provided, illustrating the scraping process performed as the inhalation progresses. FIG. 2a shows the state prior to inhalation where the rotor is stalled and the flexible arm 21 remains adjacent to the rotor 20. As the rotor 20 rotates beyond a predefined threshold, the scraping means which is the distal end 24 of said flexible arm 21 comes into contact with the drug-containing volume 19 as shown in FIG. 2b. As described above, the drug-containing volume 19 is a low-profile volume, and thus the distal end 24 of the flexible arms 21 can pass over said drug-containing volume 19 while scraping, rubbing, rattling, brushing, or otherwise removing particles from its upper surface, thereby releasing fine powder into the air stream. Advantageously, by controlling the flow parameters and the rotor mechanical parameters, a control on the delay and duration of the drug release can be achieved, resulting in synchronization with the inhalation cycle, and ensuring the delivery of the drug in accordance with the inhalation characteristics. Additionally, by controlling the mechanical properties of the rotor 20 and in particular the mass and flexibility of the flexible arms 21, the properties of the scraping means 24 and the properties of the drug-containing volume 19, the characteristics of the powder generated and inhaled can be controlled. As stated above, said drug-containing volume 19 belongs to the group including (a) a powder formed into a solid matrix and impregnated or otherwise attached to the surface of the flow chamber 23, or (b) deposition, (c) a powder maintained behind a porous mesh or (d) a powder embedded and/impregnated into a carrier like a textured material such as a fabric or other fibrous material, said carrier being attached to the spine 11. FIG. 2d illustrates the case where the drug-containing volume 19 is a powder formed into a solid matrix, and all of said powder has been scraped away by the distal end 24 of the flexible arms 21. In the case where a carrier or mesh is used to hold the powder, said carrier (i.e., the mesh, of the textured or fibrous base) will remain, even after the powder has been released.

Numerous means are known in the art to form a solid matrix or pellet from a powder, said matrix or pellet being suitable for use as the drug-containing volume 19 shown above. For example, budesonide can be compacted with lactose by drying the powder and then filling it into the dust reservoir of a pneumatic press. Said drying can be done for example by vacuum desiccation with additional help of silica gel. Compression may be carried out under pressure to form the appropriate drug-containing volume 19 ready for scraping.

Similarly, numerous means are known in the art for entrapping particles in or onto a suitable carrier material to form said powder-containing volume 19. Said carrier may be constructed from one or more of a wide range of natural and synthetic materials e.g., polyethylene, polypropylene, polyester, polytetrafluoroethylene or a copolymer thereof and cellulose. The materials may be in the form of non-woven fibrous materials, loose weave materials or fabrics, materials having a surface pile, films, microporous materials, micro-grooved materials, cords of twisted fibers, or any material or composite of more than one material having small surface grooves, recesses, interstices, apertures or embossed surface structures. An example of a suitable microporous material is produced by a laser drilling process and comprises a tape, web or belt having pores of suitable size to hold the fine powder. A non-woven material may contain any type and form of fibers. Formation of the non-woven material may be any suitable method, for example, combing or carding, deposition of fibers from a transport gas or fluid, or the extrusion and blowing of microfibers. The carrier may be loaded by the brushing, scraping or smearing of powdered medicament onto the carrier surface. Alternatively the carrier may be loaded by evaporation from a suspension of medicament, by precipitation from a solution of medicament or by deposition from an aerosol for example by spraying, impaction, impingement, diffusion or by electrostatic or van der Waals attractions. For example, the medicament particles may be given an intentional electrical charge immediately prior to loading. The technique of charged aerosol deposition may be complemented by the use of a carrier with an inherent electrostatic charge. In that case, the carrier should be an insulator such as polytetrafluoroethylene capable of retaining the charge. Alternatively the carrier may contain an artificial charge due to the presence of electrets. A further means of forming the solid matrix is deposition on a wall such as vapor deposition or electrostatic deposition as per U.S. Pat. No. 6,923,979.

In a further preferred embodiment, the drug-containing volume 19 is fabricated by containing the drug powder beneath a mesh attached to the spine 11 and the scraping means is a brush implemented at the distal end 24 of the extending arms 21. Said mesh may be adhered or welded to the spine such that the powder is stored between the mesh and the spine. Alternatively, said mesh may be part of a sachet which holds the powder. The effect of brushing, rubbing or beating this mesh by impact of the distal end 24 of the flexible arms 21 is similar to the beating of a carpet in that the fine powder is released. Alternatively (or additionally) the impact may serve to vibrate the mesh, producing a similar result. Said vibration may be due to pressure waves generated by the rotor, or interference of the rotor with a static object so as to produce sound waves and/or shock waves. Advantageously, and as demonstrated in US published patent application 20040107963, the mesh can serve to minimize agglomeration of those particles going through it; so this arrangement is effective for disaggregating powder particles. Suitable materials for the mesh include any type of woven or non-woven material or perforated film. Especially suitable are the Cryovac™ perforated films available from Sealed Air Corporation (Duncan, S.C.). One such series of films is the SM series of perforated hydrophobic polypropylene copolymer film. The preferable size of the perforations is from 20 microns to 100 microns; most favorably between 20 microns and 40 microns. A number of shapes of mesh coverings are possible, ranging from a slightly convex shape protruding above the level of the spine to a more sock-like shape. The directing of at least part of the air flow through the device via the mesh may further enhance the performance of the powder particle release. Furthermore, the drug-containing volume may be in communication with a piezoelectric material or other vibrating member serving to actively facilitate the dispersion and release of dry powder drug formulations during inhalation, as per PCT patent publication WO 01/68169A1, U.S. Pat. No. 6,889,690, and U.S. Pat. No. 6,152,130 hereby all incorporated by reference. Still further, the mesh covering of the drug-containing volume may be electrically conducting. In this case, said mesh may be electrically charged in order to influence the powder particles beneath it.

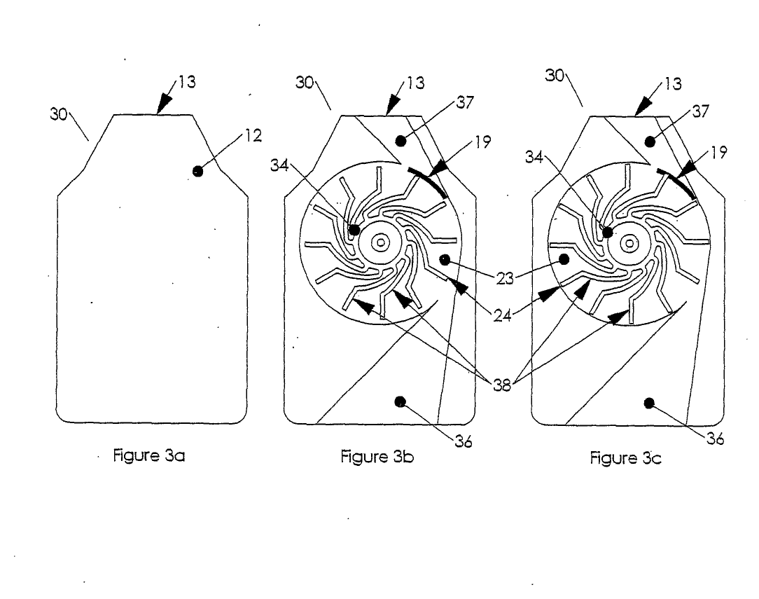

Referring now to FIG. 3, a further preferred embodiment of the present invention is shown. FIG. 3a provides a planar view of a device 30 having, as above, the shape of a credit card. The body of the device 30 comprises a spine 31 (not visible in FIG. 3a) and a film 12 covering over the air flow path in said spine 11. FIG. 3b shows the same planar view as per FIG. 3a, but with the film 12 removed and the rotor 34 exposed. This rotor 34 employs a paddle wheel design, said paddle wheel rotor 34 being accommodated in its housing within the spine 11 as per FIGS. 1 and 2. Note that this design involves the use of a tangential inlet 36 and a tangential outlet path 37. Accordingly, the air exits from the periphery of the flow chamber 23 as it does in the embodiment shown in FIG. 1. The blades 38 of the rotor 34 have the capacity to expand outward as the rotor 24 rotates, causing their distal end 24 to reach the drug-containing volume 19 as shown in FIG. 3c and serve as the scraping means. By controlling the flow parameters, and the rotor's mechanical parameters, control of the delay and duration of the drug release can be achieved, resulting in synchronization with the inhalation cycle. Also in this embodiment, by controlling the mechanical properties of this rotor 34, and in particular the mass and flexibility of the blades 38 and the properties of the scraping means on the distal ends 24 of the rotor 20 and the properties of the drug-containing volume 19, the characteristics of the powder generated and inhaled can be controlled. Advantageously, the overall result of this mechanism is the provision of a breath-powered, controllably-delayed drug delivery device which can be sustained during the breath of the patient, and prevent premature delivery of the dose at the start of inhalation.

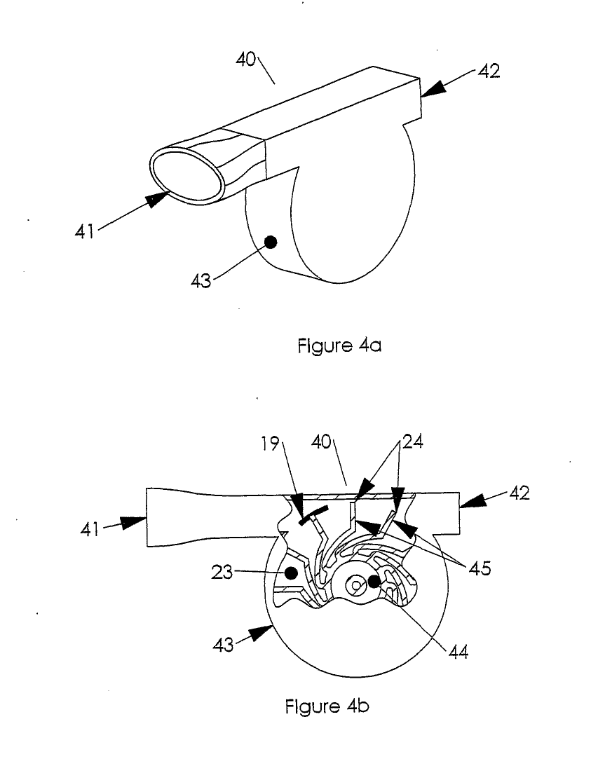

While the above embodiments describe credit-card shape designs, it will be obvious to one skilled in the art that a number of device designs are possible, including a range of solutions for drug-containing volume arrangements, and loading and replacing solutions for said volume. For example, the device may be in the shape of a prism, a disk, an oval, or use the form-factor of existing, conventional hand-held inhalers; providing only that the internal volume is sufficient to allow the breath-powered scraping or rubbing action to liberate the fine powder as described above. For example, FIG. 4a provides a illustration of a larger inhaler device 40 comprising a housing 43, containing an air inlet 42 and a mouthpiece 41. A cut-away section in FIG. 4b then shows that this embodiment further comprises a larger paddle wheel design of rotor 44 whose blades 45 extend to scrape the fine powder from the powder-containing volume 19 during the inhalation. Here also, as in both previous embodiments, the air exits from the periphery of the flow chamber 23 of the device.

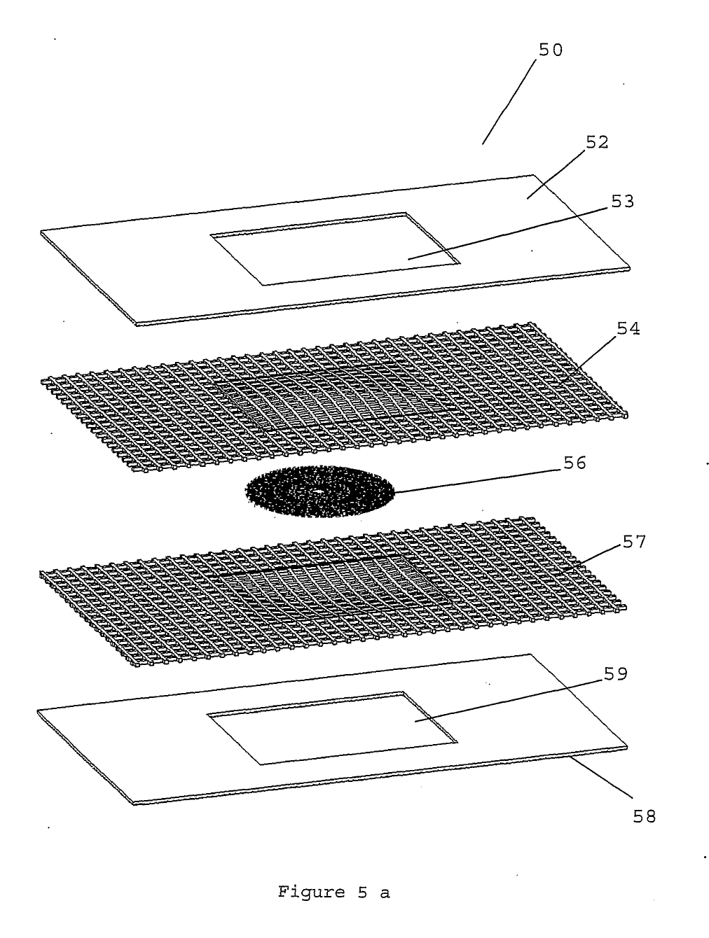

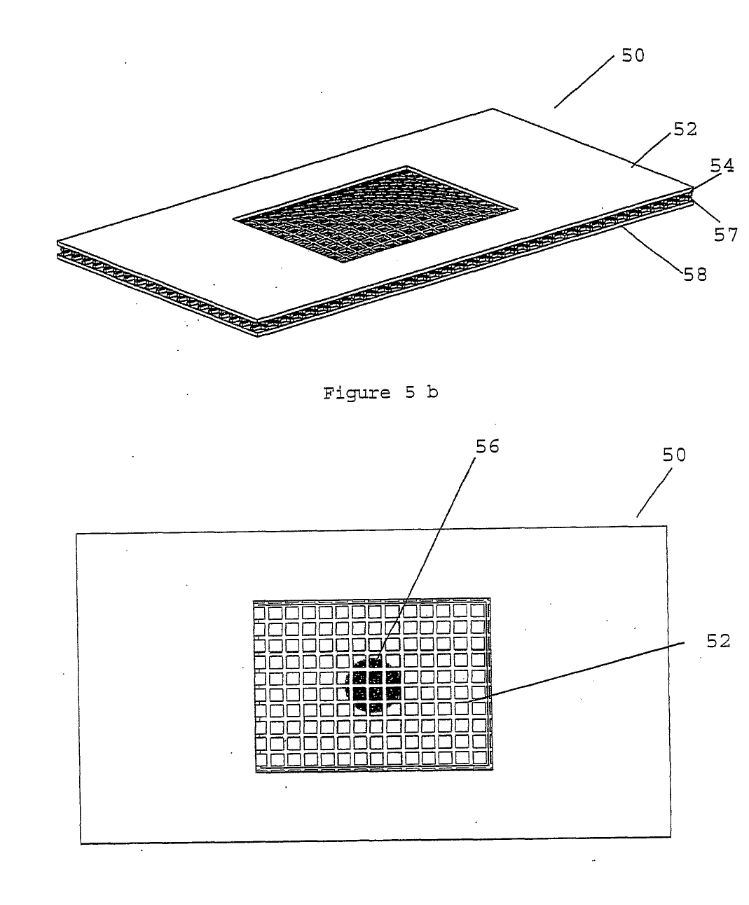

As described above, the drug-containing volume employed by the present invention can comprise a free flowing drug powder constrained within a mesh; said volume being beaten or otherwise scraped or vibrated by the action of the rotor blades. FIG. 5 illustrates a preferred embodiment of a sandwich-type design of a suitable drug-containing volume for use according to this approach. FIG. 5a provides an exploded diagram of said sandwich-type embodiment 50, said drug-containing volume 50 comprising an upper frame 52, and a lower frame 58, said frames enclosing an upper mesh 54 and a lower mesh 57; where the drug powder 56 is located between the two meshes. In a preferred embodiment, the frames (52, 58) are fabricated from a plastic material such a PVC, PET or PE together with an adhesive layer; such that applying pressure and/or heat to the outer frame of the sandwich serves to seal the sandwich as a whole; the adhesive penetrating through the mesh layers. The upper frame 52 has a window 53 cut into it, and the lower frame also has a window 59 cut into it, so that the mesh/drug area exposed by said windows will be accessible to the airflow flowing through the inhaler of the present invention. Suitable materials for the meshes include both the polymeric materials listed above and metal meshes such as MicroMesh® electroformed meshes from Precision Eforming LLC, Cortland, N.Y., USA, and mesh or TPS sieve material from Tecan Ltd., Weymouth, Dorset, UK. Referring now to FIG. 5b, an isometric view of the assembled drug-containing volume 50 according to this sandwich structure is shown.

Referring now to FIG. 5c, a planar view of this type of drug-containing volume 50 enables the drug powder particles 56 to be seen, entrapped between the mesh layers. Whereas the dry powder drug powder particles are typically less than 5 microns in diameter and preferably between 1 and 3 microns, the holes in the meshes are approximately 10-20 microns in size. This relationship causes the dry powder particles 56 to essentially remain trapped in place until the drug-containing volume 50 of this preferred embodiment is beaten, scraped or otherwise vibrated. When such beating takes place, a sieving of the particles 56 takes place, causing them to disaggregate and exit from the mesh.

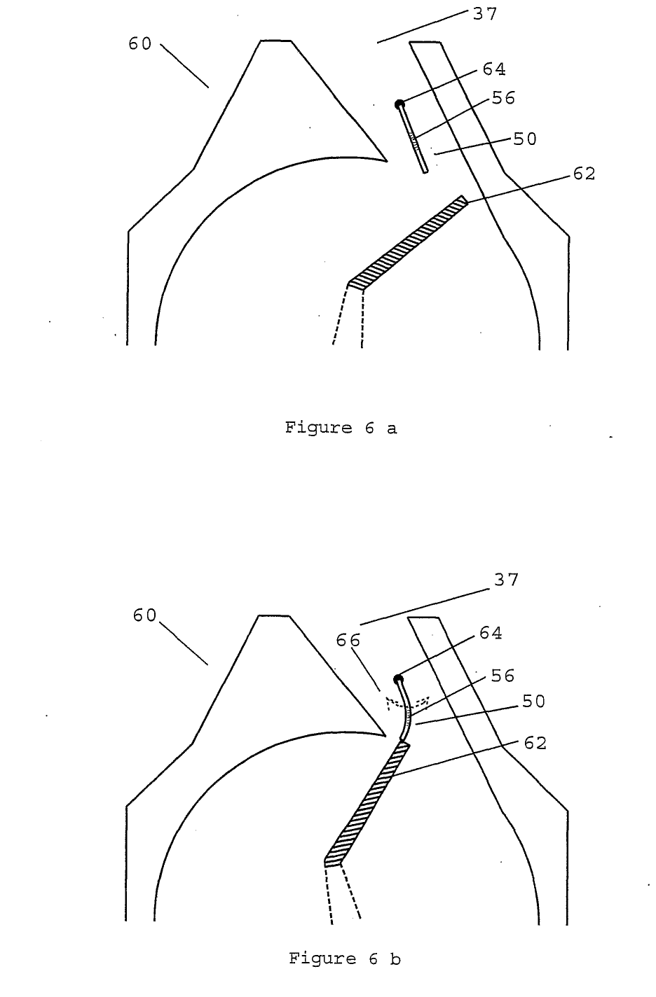

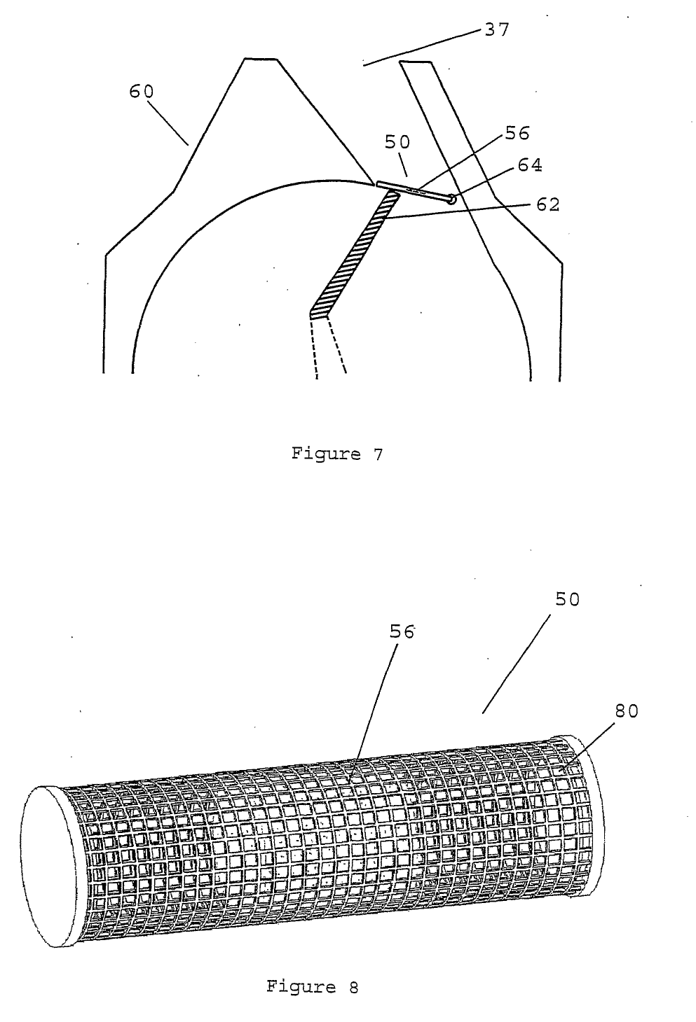

Referring now to FIG. 6, a preferred configuration of said sandwich-type drug-containing volume 50 within the inhaler is shown. FIG. 6a illustrates the location of the volume 50 within the air outlet path 37 of the inhaler 60. In a preferred embodiment, said drug-containing volume 50 is anchored at one of its ends to a rigid anchor 64; preferably by means of a slit in said anchor 64, such that the rest of the volume 50 is free to move when struck or scraped. The inhaler embodiment 60 shown is similar to that of FIG. 3 above; with the difference that the turbine blade tips 62 are arranged such that, as the turbine rotates, said tip 62 is positioned so that it will make contact with the drug-containing volume 50 each time that the tip 62 reaches said drug-containing volume 50. FIG. 6b illustrates a preferred embodiment of what happens as the turbine blade tip 62 reaches the drug-containing volume 50. Due to the flexibility of this volume 50, the blade tip 62 knocks the volume 50 sideways so that the tip 62 can continue in its rotation. In this manner, the drug-containing volume 62 receives a beating and/or scraping each time a rotor tip 62 makes contact with it. Each said beating/scraping action causes some of the drug particles 56 to be beaten out of drug-containing volume 50 and thus this beating action is the entraining action of the inhaler 60. The thus entrained drug powder particles 66 are swept up into the airflow through the air outlet path 37 and inhaled. Advantageously, this entraining action disaggregates the drug powder particles 56 by a sieving action as they exit the mesh. Furthermore, the proximity of this action to the outlet of the device and the high speed of the airflow ensure that said drug powder particles 66 have very limited opportunity to become aggregated before they are inhaled. Referring now to FIG. 7, a further preferred embodiment of the inhaler described in FIG. 6 is provided. In this embodiment the orientation of the drug-containing volume 50 is altered to be largely parallel to the air outlet path 37, so as to increase the extent to which the air flow passes through the drug-containing volume 50.

In both of the preferred embodiments shown in FIGS. 6 and 7, the rate of release of the drug can be controlled by altering parameters of the meshes used. Thus a mesh with smaller holes and/or smaller open area will constrain the powder to being entrained more slowly (i.e. requiring more blows by the turbine blades), and a mesh with larger holes and/or a larger open area will enable the drug release to be speeded up.

Referring now to FIG. 8, a further preferred embodiment of the drug-containing volume 50 is shown, in which the sandwich type enclosure for the drug powder particles 56 is replaced with a cylinder type one. However the operating principle is the same; i.e. the beating on this volume causes the powder particles to be entrained in the air flow. This preferred embodiment can of course also be configured within the inhaler of the present invention as shown in FIGS. 5 and 6 above. It will be obvious to one skilled in the art that any other design of drug-containing volume 50 in which (a) dry powder particles are enclosed, and (b) where a mesh portion of the outer wall of the volume allows particles to exit the volume when the drug-containing volume is beaten or scraped; is also included within the scope of the present invention. Accordingly, further embodiments of said drug-containing volume 50 include pyramid, diamond, cubic, spherical and oval shapes, etc. To the extent that said shapes have a significant internal volume, the airflow through them can create turbulence within said volume and thereby further enhance the disaggregation action. A still further preferred embodiment (not shown) is a candy-on-a-stick design, where the “candy” contains the drug powder and the “stick” serves either as the part which is contacted by the rotor or as the part attached to an anchor.

Dry powder drug particles are typically hygroscopic and thus it is important to enclose any and all of the types of drug-containing volumes described above within some form of blister arrangement. Such blisters typically comprise an aluminum outer foil and this may either overlay the solid matrix forms shown above, or be attached as an outer layer on the powder-containing embodiments. Numerous mechanical arrangements may be made to remove said aluminum outer foil directly before the inhaler of the present invention is used by the patient, for example by pulling out said foil via the air inlet or outlet. In the case where a particulate filter is present in the air outlet, then the air inlet is preferred. In a multi-dose embodiment or any other embodiment where the (next) drug-containing volume is inserted manually, the arrangement may be such that the insertion action causes the foil to be stripped away. The inhaler of the present invention may also or alternatively be sealed externally by such a foil, preferably with a controlled atmosphere inside the device. In a further preferred embodiment, the two types of seal (internal and external) may be used in a connected manner, such that the removal of the external seal automatically removes the foil sealing the drug-containing volume as well.

In dry-powder inhalers it is important to provide, patient feedback so that it can be verified whether a complete dose has been taken. One of the advantages of the device of the present invention is that it is easy to verify optically whether the full dose has been given. Placing a window in the housing that enables the drug-containing volume to be seen can provide a simple proof-of-administration. At its simplest, the drug will have a different color to the background, so the change in color (or absence of that color) shows that the dose has been completely administered. Note that the efficiency of the device of the present invention remains the same whether the dose has been taken in single breath or in multiple breaths and so said proof-of-administration is an important tool for the patient to know when he has inhaled the complete dose, in particular for patients with limited inhalation capacity.

Whereas in some of the above embodiments, the scraping means extend out from the rotor toward the drug-containing volume during use, in an alternative embodiment, the scraping means on the rotor assembly could remain at a fixed distance from the axis, while the drug-containing volume is spring-loaded to press forward into said scraping means or otherwise forced toward the rotor. For example a special mechanism can advance the drug toward a rotor in response to the pressure in the chamber, the speed of the rotor or the rotations of the rotor. In a further preferred embodiment the drug-containing volume is located on the rotor assembly and the scraping means is attached to a fixed point within the flow chamber. In all these embodiments the interaction between the drug-containing volume and the scraping means occurs on rotation of the rotor, as the drug-containing volume and the scraping means are brought into mutual contact when one moves towards the other or as both move towards each other.

In the above preferred embodiments, the rotors can be made from injection-molded thermoplastic materials such as polyurethane, Polyacetal or polycarbonate, or alternatively from sheet metal spring materials. The outlet filter (not shown) can be either be an integrally-formed part of the device housing, or be a separate component such as a Porex™ piece (from Porex Corporation, Fairburn, Ga., USA) or a non-woven mesh.

The inhaler device of the present invention may be provided in either single-dose or disposable or multiple-use embodiments. For example, co-pending application PCT/IL2006/000647 describes embodiments where the drug-containing volume is mounted on a movable element such as a carousel, such that one dose is presented towards the free-powder releasing means at a time. Then, by rotating the carousel, the next drug dose can be accessed. Alternative multi-use variants of the approach are also described in said co-pending application (herein incorporated by reference), including where the drug-containing volume is in the shape of a bar that can be incrementally advanced. Due to the inexpensive nature of the designs employed above, the device presented can be a disposable one, whether intended for multiple-use or single use. Alternatively, the drug-delivery device of the present invention can be designed so that the drug-containing volume can be replaced by the user, thus making the device a permanent multiple-use device. Further, it will be obvious to one skilled in the art that a number of drugs can be inhaled simultaneously using the device of the present invention, whether by employing a multiplicity of drug-containing volumes where different volumes contain different drugs, or by means of mixing a plurality of drugs within any given drug-containing volume. Additionally, where a “magazine” of drug-containing volumes is used, each of said volumes may comprise a different drug or different drug combination. Advantageously, said arrangement enables the sequential administration of a number of drugs.

It should also be apparent that the device of the present invention can further incorporate a number of standard drug-dosing device components or functions known in the art. These elements include a child-proof mechanism to protect against inadvertent activation by a child; a counter display showing the number of inhalations, shipping seals, air-tight resealing plugs, etc.

Many medications suitable for delivery by the inhaler device of the present invention as sensitive to moisture and thus, in a preferred embodiment, the device may be sealed by a tape such as a metalized tape covering the outlet and air inlet(s). In a preferred embodiment said tape will have a tap for easy removing before using the device. Additionally, the device as whole may be packaged in a non-permeable film for additional protection.

It will be obvious to those skilled in the art that the device of the present invention can be part of a more complex system. For example the device can be connected to a flow spacer that is a common feature in this field, or can be incorporated as a cartridge in a more complex inhalation system.

Suitable medicaments for use in the invention include any drug or drugs which may be administered by inhalation and which is either a solid or may be incorporated in a solid carrier. Suitable drugs include those for the treatment of respiratory disorders, e.g., bronchodilators, corticosteroids and drugs for the prophylaxis of asthma. Other drugs such as anorectics, anti-depressants, anti-hypertensive agents, anti-neoplastic agents, anti-cholinergic agents, dopaminergic agents, narcotic analgesics, beta-adrenergic blocking agents, prostoglandins, sympathomimetics, tranquilizers, steroids, vitamins and sex hormones may be employed. Exemplary drugs include: Salbutamol, Terbutaline, Rimiterol, Fentanyl, Fenoterol, Pirbuterol, Reproterol, Adrenaline, Isoprenaline, Ociprenaline, Ipratropium, Beclomethasone, Betamethasone, Budesonide, Disodium Cromoglycate, Nedocromil Sodium, Ergotamine, Salmeterol, Fluticasone, Formoterol, Insulin, Atropine, Prednisolone, Benzphetamine, Chlorphentermine, Amitriptyline, Imipramine, Cloridine, Actinomycin C, Bromocriptine, Buprenorphine, Propranolol, Lacicortone, Hydrocortisone, Fluocinolone, Triamcinclone, Dinoprost, Xylometazoline, Diazepam, Lorazepam, Folic acid, Nicotinamide, Clenbuterol, Bitolterol, Ethinyloestradiol and Levenorgestrel. Drugs may be formulated as a free base, one or more pharmaceutically acceptable salts or a mixture thereof.

A dry-powder inhaler is described above. Various details of the invention may be changed without departing from its scope. Furthermore, the foregoing description of the preferred embodiment of the invention and the best mode of practicing the invention are provided for the purpose of illustration only and not for the purpose of limitation; the invention being defined by the claims.

What is claimed is:

1. A dry-powder, breath-powered inhaler device comprising at least one air inlet, a flow chamber and an air outlet leading to a mouthpiece, said flow chamber further comprising at least one drug-containing volume and at least one free-powder releasing means for entraining powder into air flow through said device and a rotor assembly comprising an axis and at least one rotor blade; wherein the inhalation action of the patient applied at said air outlet causes air to enter said at least one air inlet such that said air interacts with said at least one rotor blade causing said rotor to rotate and thus generate breath-driven, relative motion between said at least one drug-containing volume and said at least one free powder releasing means, such that fine free particles of powder are released from said drug-containing volume; the arrangement being such that the air exits said flow chamber via the periphery of said flow chamber, carrying out said fine particles to said outlet to be inhaled by the patient.

2. The inhaler device of claim 1, wherein said free-powder releasing means are selected from the group consisting of scraping means, a brush and beater means.

3. The inhaler device of claim 2 where said scraping means is located at the distal end of said rotor blade, said blade gradually extending outwards as said rotor rotates.

4. The inhaler device of claim 2 where said scraping means is located at the distal end of an arm attached to said rotor blade, said arm gradually extending outwards as said rotor rotates.

5. The inhaler device of claims 3 and 4 where the drug-containing volume is fixed within the flow chamber.

6. The inhaler device of claim 1 where said drug-containing volume comprises a medicinal agent belonging to the group including drugs, vaccines and other therapeutic agents

7. The inhaler device of claim 1 where said drug-containing volume comprises fine powder particles belonging to the group including powder, micronized powder and microspheres.

8. The inhaler device of claim 1 where said drug-containing volume is formed by means including compression into a solid matrix, impregnation onto a surface, embedding in a carrier, holding behind a mesh and deposition on a surface.

9. The inhaler device of claim 1 where said drug-containing volume is a solid drug matrix and the free-powder releasing means is a scraper which successively scrapes powder particles from its surface.

10. The inhaler device of claim 1 where said drug-containing volume comprises drug powder located behind a porous mesh, and a free-powder releasing means successively impacts said volume so as to cause powder particles to be released through said mesh

11. The inhaler device of claim 1 further comprising a particle filter located between said flow chamber and said outlet to ensure that large particles are not inhaled.

12. The inhaler device of claim 1 where said device is shaped likes credit-card

13. The inhaler device of claim 1 where the shape of said device belongs to the group including cylinders, prisms, disks ovals, and conventional hand-held inhalers.

14. The inhaler device of claim 1 where the drug-containing volume is located on the rotor assembly and the free-powder releasing means is connected to the flow chamber.

15. The inhaler device of claim 1 where the drug-containing volume extends toward the free-powder releasing means.

16. The inhaler device of claim 1 where the free-powder releasing means extends toward the drug-containing volume.

17. The inhaler device of claim 1 where the rattling of the drug-containing volume is achieved by part of the rotor assembly interacting with a non-flat pan of the flow chamber.
